(19) United States
(12) Patent Application Publication
(10) Pub. No.: US 2019/0351261 A1
(43) Pub. Date: Nov. 21, 2019

(54) SELECTIVE RESAMPLING DURING NON-INVASIVE THERAPY

(71) Applicant: Yoav LEVY, Hinanit (IL)

(72) Inventor: Yoav LEVY, Hinanit (IL)

(21) Appl. No.: 15/983,530

(22) Filed: May 18, 2018

(51) Int. Cl. A61N 7/02; A61B 90/00; A61B 5/055

(52) U.S. Cl. CPC A61N 7/02; A61B 90/37; A61N 2007/0078; A61B 2090/374; A61B 5/055

(57) ABSTRACT

During a focused-ultrasound or other non-invasive procedure, regions of change within a target region are monitored, and images of the target region are updated with partial images encompassing only the regions of change.

### TECHNICAL FIELD

The present invention relates, in general, to monitoring of changing or moving tissue or organs, and, in particular, to monitoring thereof during non-invasive therapy.

### BACKGROUND

Tissue, such as a benign or malignant tumor or blood clot within a patient's skull or other body region, may be treated invasively by surgically removing the tissue or non-invasively by using, for example, thermal ablation. Both approaches may effectively treat certain localized conditions within the body, but involve delicate procedures to avoid destroying or damaging otherwise healthy tissue. Unless the healthy tissue can be spared or its destruction is unlikely to adversely affect physiological function, surgery may not be appropriate for conditions in which diseased tissue is integrated within healthy tissue.

Thermal ablation, as may be accomplished using focused ultrasound, has particular appeal for treating diseased tissue surrounded by or neighboring healthy tissue or organs because the effects of ultrasound energy can be confined to a well-defined target region. Ultrasonic energy may be focused to a zone having a cross-section of only a few millimeters due to relatively short wavelengths (e.g., as small as 1.5 millimeters (mm) in cross-section at one Megahertz (1 MHz)). Moreover, because acoustic energy generally penetrates well through soft tissues, intervening anatomy often does not impose an obstacle to defining a desired focal zone. Thus, ultrasonic energy may be focused at a small target in order to ablate diseased tissue without significantly damaging surrounding healthy tissue.

An ultrasound focusing system generally utilizes an acoustic transducer surface, or an array of transducer surfaces, to generate an ultrasound beam. The transducer may be geometrically shaped and positioned to focus the ultrasonic energy at a “focal zone” corresponding to the target tissue mass within the patient. During wave propagation through the tissue, a portion of the ultrasound energy is absorbed, leading to increased temperature and, eventually, to cellular necrosis—preferably at the target tissue mass in the focal zone. The individual surfaces, or “elements,” of the transducer array are typically individually controllable, i.e., their phases and/or amplitudes can be set independently of one another (e.g., using a “beamformer” with suitable delay or phase shift in the case of continuous waves and amplifier circuitry for the elements), allowing the beam to be steered in a desired direction and focused at a desired distance and the focal zone properties to be shaped as needed. Thus, the focal zone can be rapidly displaced and/or reshaped by independently adjusting the amplitudes and phases of the electrical signal input into the transducer elements.

However, because the human body is flexible and moves even when a patient is positioned to keep still (due to respiration, for example, or small involuntary movements), treatment delivered as multiple sonications over time—even when delivered within seconds of each other—may require interim adjustments to targeting and/or to one or more treatment parameters. Compensation for motion is thus necessary to ensure that the ultrasound beam remains focused on the target and does not damage the surrounding healthy tissues.

Accordingly, an imaging modality, such as magnetic resonance imaging (MRI), may be used in conjunction with ultrasound focusing during non-invasive therapy to monitor the locations of both the target tissue and the ultrasound focus. Generally, an MRI system 100, as depicted in FIG. 1, includes a static-field magnet 102, one or more gradient-field coils 104, a radio-frequency (RF) transmitter 106, and an RF receiver (not shown). (In some embodiments, the same device is used alternately as RF transmitter or receiver.) The magnet includes a region 108 for receiving a patient 110 therein, and provides a static, relatively homogeneous magnetic field over the patient. Time-variable magnetic field gradients generated by the gradient-field coils 104 are superposed with the static magnetic field. The RF transmitter 106 transmits RF pulse sequences over the patient 110 to cause the patient's tissues to emit a (time-varying) RF response signal, which is integrated over the entire (two- or three-dimensional) imaging region and sampled by the RF receiver to produce a time series of response signals that constitute the raw image data. This raw data is passed on to a computation unit 112. Each data point in the time series can be interpreted as the value of the Fourier transform of the position-dependent local magnetization at a particular point in k-space (i.e., wavevector space), where the wavevector k is a function of the time development of the gradient fields. Thus, by inverse Fourier-transforming the time series of the response signal, the computation unit 112 can reconstruct a real-space image of the tissue (i.e., an image showing the measured magnetization-affecting tissue properties as a function of spatial coordinates) from the raw data. The real-space magnetic-resonance (MR) image may then be displayed to the user. The MRI system 100 may be used to plan a medical procedure, as well as to monitor treatment progress during the procedure. For example, MRI may be used to image an anatomical region, locate the target tissue (e.g., a tumor) within the region, guide the beam generated by the ultrasound transducer 114 to the target tissue, and/or monitor the temperature in and surrounding the target tissue.

While MRI enables the beneficial use of image-guided systems, such as MRI-guided focused-ultrasound (MRgFUS) systems, in a variety of different treatment scenarios, in many cases the imaging rate (i.e., the rate at which successive MRI images may be acquired) lags behind the rate at which one or more characteristics of the target changes. For example, during a focused ultrasound treatment or other exposure to therapeutic energy, the location of the target or the temperature of the target may rapidly change and may be revealed only as large discontinuous jumps from one full MRI scan to another. This inability to track changes in the target at a sufficiently fine timescale may result in significant inefficiencies in the treatment process (as the treatment may need to be stopped and reset to account for the change) or even dangerous exposure to treatment energy of the target or to untargeted tissue.

Accordingly, there is a need for improved image-acquisition approaches that facilitate monitoring of the target, e.g., in real time during treatment, which are responsive to one or more rapidly changing characteristics of the target and/or the surrounding tissue.

### SUMMARY

In various embodiments, the present invention provides systems and methods for monitoring one or more rapidly changing characteristics of a target (e.g., a treatment target) or other objects of interest in a region of interest in real time during an image-guided treatment procedure. In various embodiments, a full (or “complete”) image of a treatment area is acquired prior to initiation of treatment and utilized as a baseline reference image. After initiation of treatment, one or more full comparative images of the treatment area are acquired, and the regions of the treatment area changing most rapidly are identified via comparison to the baseline reference image. As will be appreciated, the specific changes identified via image comparison will relate to the properties of the target or the target area that are being monitored, and/or to the treatment procedure being performed. For example, position shifts and/or temperature changes of a treatment target may be monitored based at least in part on the acquired images. After identification of the regions of the treatment area in which changes are occurring most rapidly, the imaging process is optimized via acquisition of only partial image data corresponding to those regions. The partial image data may be combined with image data previously acquired only in the full comparative images to reconstruct new full images in which only the rapidly changing portions are updated. In this manner, relatively slow imaging modalities (e.g., MRI) may be utilized during real-time treatment procedures even when the rate of acquisition of full images of the treatment area is slower than the rate of change occurring within the treatment area. In short, embodiments of the current invention significantly reduce the image acquisition and processing time required during treatment by acquiring and processing only partial image data (i.e., partial raw k-space data and/or real-space sub-images) that include the regions(s) of the treatment area in which changes are detected, thus facilitating real-time monitoring of the treatment area (and obj ect(s) of interest therewithin) with limited delay.

In various embodiments, it is the k-space data that are only partially updated based on the degree of change thereto over time. For example, entire rows, entire columns, or portions of various rows and/or columns may be excluded from updating so that previously obtained k-space pixels are re-used in one or more new k-space data frames. In accordance with embodiments of the invention, the specific data regions corresponding to areas of rapid change are identified from successive full k-space scans, and the identified regions are updated during treatment using partial k-space scans. In this manner, embodiments of the invention contrast with techniques in which predetermined k-space data portions (e.g., low spatial frequencies in k-space) are repeatedly acquired in or excluded from partial scans irrespective of changes occurring therein or without detection or monitoring of changes occurring in the treatment area. In other embodiments, rather than or in addition to periodically examining full scans to identify the regions that do not require updating, pixels within the regions that would otherwise be excluded from updating altogether can instead be sampled at a lower resolution for comparative purposes in order to verify that they still are not changing rapidly. In this way, the benefits of a lower sampling rate can be obtained without re-analyzing the full image, since in the static regions, a smaller number of pixels are monitored over time and compared against earlier versions.

In various embodiments of the present invention, only areas of a full comparative image in which the rate of change of a monitored parameter is high based on some measure (as described more fully herein) are updated in one or more successive imaging scans. In various embodiments, change rates within the K space may be compared to multiple different thresholds, and different areas of the K space may be updated via targeted scans at different update frequencies, depending upon which thresholds are exceeded. For example, an area of K space exhibiting only a moderate rate of change in the initial image comparison (i.e., a rate of change exceeding only a first threshold) may be updated less frequently (e.g., every 2-5 scans) when compared to areas of K space exhibiting larger rates of change (i.e., rates that exceed a second threshold larger than the first threshold), which may be updated more frequently (e.g., during each scan).

In various embodiments, one or more full comparative K space may be acquired periodically during the treatment procedure, and the partial image areas to be updated in subsequent partial scans may be updated based on the new full scans. In this manner, embodiments of the invention ensure that the most relevant image areas are being updated even during lengthy treatment procedures or during procedures in which one or more characteristics may change at different rates during the procedure.

The various images utilized in embodiments of the present invention may be stored as raw k-space image data and/or reconstructed real-space image data; however, rates of change may be determined based on k-space as acquisition is performed in that space.

More generally, the present invention reduces the need for image resampling in any region of interest of a human or non-human subject or a model (e.g., a “phantom”) used to test, calibrate, or refine a therapeutic system. The result is to improve real-time monitoring and reduce the computational load in therapeutic and non-therapeutic settings.

In an aspect, embodiments of the invention feature a system for imaging a target region including, consisting essentially of, or consisting of a feature (e.g., an anatomical feature, a treatment target, a non-treatment target, a phantom such as an imaging phantom, etc.) therewithin. The system includes, consists essentially of, or consists of an imaging apparatus for acquiring images and a computation unit. The imaging apparatus may be operable in conjunction with a treatment apparatus. The imaging apparatus is configured to acquire and computationally store (i) a baseline k-space image of the target region, (ii) a comparative k-space image of the target area during an operational sequence, and (iii) one or more new k-space images each encompassing only a portion of the target region during the operational sequence. The computation unit is configured to (i) computationally compare the comparative k-space image with the baseline k-space image to identify one or more first image regions of the k space associated with a changed characteristic within the target region, the comparative k-space image including, consisting essentially of, or consisting of (a) the one or more first image regions and (b) a remaining image region, and (ii) cause the imaging apparatus to acquire a new k-space image by sampling only in the one or more first image regions.

Embodiments of the invention may include one or more of the following in any of a variety of different combinations. The new k-space may include, consist essentially of, or consist of pixels corresponding to the newly sampled one or more image regions and information based at least in part on previously sampled pixels corresponding to the remaining image region. The computation unit may be configured to computationally reconstruct a real-space image from a comparative k-space image and/or from a new k-space image. The imaging apparatus may include, consist essentially of, or consist of an MRI apparatus. The treatment apparatus may include, consist essentially of, or consist of one or more ultrasound transducers. The changed characteristic within the target region may include, consist essentially of, or consist of a pixel value. The computational unit may be configured to steer and/or modulate an energy beam (e.g., a therapeutic energy beam) based on the new k-space image and/or a real-space image computationally reconstructed therefrom. The energy beam may include, consist essentially of, or consist of a focused ultrasound beam. The operational sequence may include, consist essentially of, or consist of exposure of a target other than the feature (e.g., to an energy beam or a therapeutic energy beam). The computational unit may be configured to shape and/or steer an energy beam onto the target so as to avoid the feature based on the new k-space image and/or a real-space image computationally reconstructed therefrom. The energy beam may include, consist essentially of, or consist of a focused ultrasound beam. The computation unit may be configured to (i) identify a plurality of first image regions of the k space associated with the changed characteristic within the target area, and (ii) sample in the first image regions at a frequency, for each first image region, based at least in part on a magnitude of the change in the characteristic therein. The one or more first image regions may be identified at least in part by estimation based on at least one previous k-space image.

In another aspect, embodiments of the invention feature a method for imaging, during an operational sequence, a target region including, consisting essentially of, or consisting of a feature. A baseline k-space image of the target region is acquired. Thereafter, during the operational sequence, steps (a), (b), and (c) are performed. In step (a), a comparative k-space image of the target region is acquired. In step (b), the comparative k-space image is computationally compared with the baseline k-space image to identify one or more first image regions of the comparative k-space image having a changed characteristic. The comparative k-space image includes, consists essentially of, or consists of (i) the one or more first image regions and (ii) a remaining image region. In step (c), a new k-space image is subsequently acquired by sampling only the one or more first image regions. The new k-space image including, consisting essentially of, or consisting of pixels corresponding to the newly sampled one or more first image regions and additional pixel values based at least in part on previously sampled pixels corresponding to the remaining image region.

Embodiments of the invention may include one or more of the following in any of a variety of combinations. A real-space image may be computationally reconstructed from the new k-space image. The real-space image may be displayed. Step (c) may be repeated one or more times. Steps (a) and/or (b) may be repeated after repeating step (c) one or more times. The changed characteristic within the target region may include, consist essentially of, or consist of a pixel value. The baseline k-space image and/or the comparative k-space image may include, consist essentially of, or consist of full-scan MRI images. The new k-space image may include, consist essentially of, or consist of a partial-scan MRI image. The operational sequence may include, consist essentially of, or consist of exposure of the feature to an energy beam (e.g., a therapeutic energy beam). The operational sequence may include, consist essentially of, or consist of steering and/or modulating an energy beam (e.g., a therapeutic energy beam) based on the new k-space image and/or a real-space image computationally reconstructed therefrom. The energy beam may include, consist essentially of, or consist of a focused ultrasound beam. The operational sequence may include, consist essentially of, or consist of exposure of a target other than the feature (e.g., to an energy beam or a therapeutic energy beam). An energy beam may be shaped and/or steered and/or modulated onto the target region so as to avoid the feature based on the new k-space image and/or a real-space image computationally reconstructed therefrom. The energy beam may include, consist essentially of, or consist of a focused ultrasound beam. A plurality of first image regions of the k space associated with the changed characteristic within the target area may be identified. The method may include sampling in the first image regions at a frequency, for each first image region, based at least in part on a magnitude of the change in the characteristic therein. The one or more first image regions may be identified at least in part by estimation based on at least one previous k-space image.

As used herein, the term “acquiring” includes obtaining data from sensors or imagers, retrieving data from sources such as treatment images, and computing or deriving new information from existing data. Throughout the present disclosure, a “treatment sequence” may be a sequence whereby treatment (for example ultrasound treatment) is carried out or the “treatment sequence” may refer to carrying out a sequence absent any treatment being carried out. Similarly, a “treatment image” refers to an image obtained during a “treatment sequence” with treatment or absent treatment. Baseline images, comparative images, and partial images may be obtained during treatment sequences and/or with treatment or absent treatment. Accordingly, methods detailed herein optionally do not include any treatment. In various embodiments, methods detailed herein are not or do not comprise a method of treatment of the human body and/or an animal body.

As used herein, the term “substantially” mean±10%, and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 illustrates an MRI-guided focused ultrasound system in accordance with various embodiments of the invention;

FIG. 2 is a flow chart illustrating a method in accordance with various embodiments of the invention;

FIG. 3 is a block diagram illustrating an image-processing and control facility in accordance with various embodiments of the invention;

FIG. 4 is an exemplary image of a treatment area depicting patient tissue targeted for treatment in accordance with various embodiments of the invention;

FIG. 5 is a map of temperature rise within the treatment area following treatment of the tissue depicted in FIG. 4;

FIG. 6 is a map, in k-space, of the relative temperature change within the treatment area depicted in FIG. 5;

FIG. 7 is a temperature map constructed by combining a newly acquired partial image focused on the region of FIG. 6 of largest change with portions of FIG. 5; and

FIG. 8 is a temperature map constructed by combining a newly acquired partial image not encompassing the entirety of the region of FIG. 6 of largest change with portions of FIG. 5.

### DETAILED DESCRIPTION

In various embodiments, the present invention provides systems and methods for monitoring one or more rapidly changing characteristics of a target (e.g., a treatment target) or other objects of interest in a region of interest in real time during an image-guided treatment procedure. The procedure may, for example, involve the application of focused ultrasound to (i.e., the sonication of) a material, a tissue or organ for the purpose of heating it, either to necrose, ablate, or otherwise destroy the tissue if it is, e.g., cancerous, or for non-destructive treatments such as pain amelioration or the controlled inducement of hyperthermia. Ultrasound may also be used for other, nonthermal types of treatment, such as, e.g., neuromodulation. Alternatively, the procedure may use different forms of therapeutic energy, such as, e.g., radio-frequency (RF) radiation, X-rays or gamma rays, or charged particles, or involve other treatment modalities such as cryoablation.

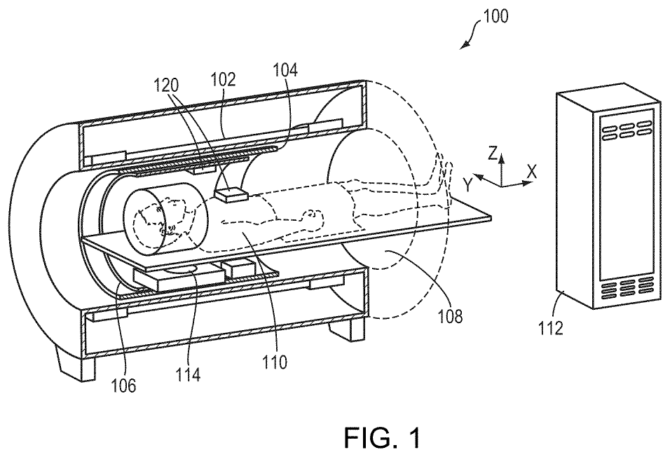

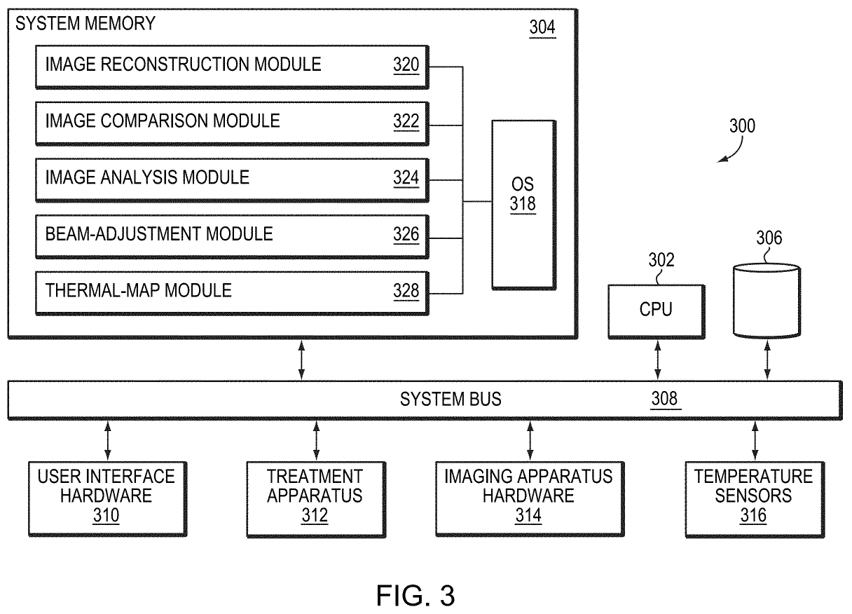

Tracking of various changing characteristics (e.g. position and/or temperature) in treatment procedures may serve to guide the therapeutic energy beam onto the target and/or around other, non-target tissues and organs, i.e., to adjust the beam focus, profile, and/or direction based on images of the affected anatomical region, which may, in some embodiments, also visualize the beam focus. MRI is a widely used technique for such image-based tracking. However, other imaging techniques, including, e.g., X-ray imaging, X-ray computed tomography (CT), or ultrasound imaging, may also be used and are within the scope of the present invention. In addition, the tracking may be achieved using one or more two-dimensional images and/or three-dimensional images. An exemplary system for implementing methods in accordance with various embodiments is an MRgFUS system, such as the one depicted in FIG. 1, with a suitable image-processing and control facility as described in detail below with reference to FIG. 3.

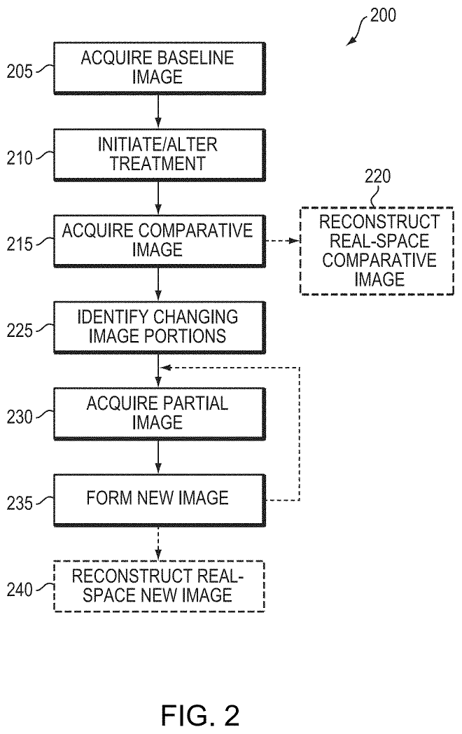

FIG. 2 depicts a method 200 for real-time tracking and image reconstruction in accordance with various embodiments of the present invention. For ease of reference, the following description only refers to target tracking; it should be understood, however, that the same techniques generally apply as well to the tracking of other organs or tissues of interest (such as organs vulnerable to damage from the therapeutic beam) or materials. In a first step 205, initial baseline image information corresponding to the treatment area (e.g., an anatomical region including a treatment target) is acquired. (As utilized herein, “image information” refers to a partial or complete real-space image or the raw data (e.g., k-space data) usable to construct such a partial or complete image.) While in various embodiments the baseline image information is acquired prior to the commencement of treatment, in other embodiments the baseline image information is acquired during treatment but upon a change in the treatment sequence (e.g., after a delay). For example, for a treatment sequence that involves multiple applications of therapeutic energy to a target, baseline image information may be acquired before one or more of the different applications. Similarly, baseline image information may be acquired prior to an alteration in the treatment sequence, e.g., a change in the intensity or location of the applied energy and/or to the volume of tissue under treatment. In MRI-based and similar methods, image information acquisition typically involves the acquisition of raw k-space data, e.g., raw k-space MR signals. In accordance with various embodiments, this raw data may be utilized for subsequent comparisons without reconstruction into a real-space image. In other embodiments, the raw k-space data may be reconstructed into a real-space image, as understood by those of skill in the art. Both k-space and real-space image data are complex-valued (i.e., have a magnitude and phase or, expressed differently, real and imaginary parts). In accordance with various embodiments of the invention, the initial baseline image information acquired in step 205 includes, consists essentially of, or consists of a full k-space scan. As used herein a “full k-space scan” or “full k-space data” connotes a complete data matrix of, for example, 16×16, 32×32, 64×64, 128×128, 128×256, 192×256, or 256×256 obtained using a standard full MM scanning procedure. In contrast, the terms “partial k-space scan” or “partial k-space data” connote a continuous or discontinuous portion of a full k-space scan or any matrix or other ordered arrangement of data containing less information than (e.g., having one or more dimensions smaller than) a full k-space scan.

In step 210, treatment of a target within the imaged treatment area is initiated or altered. As mentioned above, this step may involve either the initiation of a treatment sequence or the alteration of one or more treatment parameters during treatment. In either case, the initiation or alteration of the treatment is expected to result in or be accompanied by the change in at least one parameter (e.g., temperature, size, location, etc.) associated with the target or with another feature in the treatment area. For example, focused-ultrasound ablation of a tumor may be carried out in two or more phases: a first phase during which the central region of the tumor is targeted, and one or more subsequent phases in which the peripheral regions of the tumor are exposed to ultrasound. Since the risk to healthy tissue surrounding the tumor increases as treatment progresses, so may the need for accurate, real-time monitoring of changes within the treatment area.

In step 215, comparative image information is acquired from the treatment area after the initiation or alteration of the treatment. The comparative image information may be acquired in one or more scans of the treatment area. In various embodiments, the comparative image information corresponds to at least one full k-space scan of the treatment area. In an optional step 220, the comparative image information is computationally reconstructed to form a real-space image (e.g., via inverse Fourier transformation of k-space data).

In step 225, the comparative image information is compared with the initial baseline image information in order to identify portions thereof corresponding to changes occurring within the treatment area in response to the treatment initiated or altered in step 210. For example, changes in the position, size, or temperature of the target in response to the treatment may be encoded as changes in the acquired image information. That is, pixel values within the image information may change between the baseline scan and the comparative scan; such changing pixel values may correspond to, for example, changes in the location of the target (or a portion thereof) and/or changes in the temperature (and/or another sensed characteristic) of the target (or a portion thereof) and/or of surrounding tissue. In various embodiments, the comparative image information is compared with the baseline image information, data point by data point (e.g., k-space pixel by k-space pixel), and portions of the comparative image information in which the data has changed significantly are identified. For example, the temperature of a target, or a portion thereof, may increase in response to the initiation of treatment (e.g., the application of therapeutic energy), and the localized temperature increase will be reflected in the comparative image information. The identified portions will typically correspond to partial k-space data, e.g., one or more portions of the full k-space scan that are not necessarily contiguous in k-space.

The comparison in step 225 may be based on k-space data. Typically, the comparison is performed on a pixel-by-pixel basis, where a “pixel” refers to an element of the k-space data array, which generally stores amplitude and phase values or equivalent representation (e.g., real and imaginary of a complex number) as a function of k-space coordinates. For example, in various embodiments, the similarity between pixels of the k-space data acquired in steps 205 and 215 may be measured, and the identified portions of the k-space data correspond to those where the data is changing significantly. By “significantly” is meant that the change is sufficient that reliance on previous data would be clinically inappropriate. There are numerous quantitative and heuristic ways in which the pixels that will be updated may be identified. In one approach, the degree of change in a pixel parameter is quantified and measured to determine whether similarity falls beneath a predefined similarity threshold. That is, in some embodiments, the change in the pixel parameter between the comparative image information and the baseline image information is assessed against the similarity threshold, and only if the level of similarity falls below the threshold (which typically means, for metrics that measure the differences, i.e., the dissimilarity, between images, that the value of the metric exceeds the threshold value) is that portion of the comparative image information identified as “significantly changing.” Suitable similarity metrics include, for example, pixel intensity, cross-correlation coefficients, the sum of squared intensity differences, mutual information (as the term is used in probability and information theory), ratio-image uniformity (i.e., the normalized standard deviation of the ratio of corresponding pixel values), the mean squared error, the sum of absolute differences, the sum of squared errors, the sum of absolute transformed differences (which uses a Hadamard or other frequency transform of the differences between corresponding pixels in the two images), or complex cross-correlation (for complex images, such as MRI images), and other techniques familiar, to those of skill in the art, in connection with image comparison and registration.

The value of the threshold—that is, the amount of change sufficient to justify re-scanning in higher rate—depends on the parameter and the application. In some embodiments, the threshold is fixed, but more typically it is defined dynamically based on the k-space data itself. For example, the threshold can be defined statistically in terms of the mean intensity, e.g., ½ or 1 standard deviation from the mean pixel intensity value (or other pixel-parameter value). The threshold can also be defined based on the maximum difference in the pixel parameter across the comparative image relative to the baseline image, e.g., 25% of the maximum difference.

Alternatively, instead of an explicit threshold, the areas that have changed the most relative to a baseline may be selected for update. For example, a defined percentage of the image information exhibiting the greatest degree of change may be identified. In one embodiment, the pixels in the comparative image can be ranked in terms of their difference from the corresponding pixels of the baseline image, and the top 25% or 50% of pixels (i.e., the 25% or 50% whose differences from the baseline image are largest) are selected for update. Statistics can be used to determine the percentage of pixels identified for updating. For example, if the parameter difference considered across all pixels in the new and previous images is bimodal, all pixels within the peak having the higher mean difference may be selected.

In various embodiments, step 225 involves the comparison of the change between the comparative image information and the baseline image information can involve multiple different thresholds or other comparison metrics each corresponding to a different amount of change (based on, e.g., one or more of the similarity metrics listed above). In this manner, portions of the image information that exhibit different rates of change may be updated at different frequencies. For example, a first portion of the image information exhibiting a lesser amount of change may be updated less frequently (e.g., every 2-10 acquisitions of partial image information) than second portion of the image information exhibiting a greater amount of change (which may be updated, e.g., during each acquisition of partial image information). Thus, for example, if the if the parameter difference considered across all pixels in the new and previous images is multimodal, the pixels corresponding to each peak can be updated at a different rate. Similarly, different thresholds can be defined, each corresponding to a different update rate. Moreover, updating can be performed on identified pixel regions (i.e., regions of a given size whose mean parameter difference is considered significant) rather than at the level of specific pixels.

In various embodiments of the present invention, if no portion of the comparative image information exhibits a rate of change that exceeds any of the relevant thresholds, then the comparative image information may be utilized as the new baseline image information, and the method may commence with the acquisition of new full-scan comparative image information. This new acquisition of comparative image information may be undertaken after a delay. In various embodiments, the duration of the delay may be related to the difference between a sensed change in the previously compared images and the change threshold; for example, the delay may be decreased as the sensed change level approaches the threshold. Embodiments of the invention tolerate the slower imaging speed involved with the acquisition of new full scans due to the lack of a rapidly changing characteristic in the treatment area. In this manner, embodiments of the invention compare full-scan image information until the magnitude of a sensed change exceeds a threshold and acquisition and use of partial image information commences.

In various embodiments, k-space data is acquired in a continuous manner, usually row by row or column by column, and thus one or more portions of the image information identified in step 225 may include, consist essentially of, or consist of one or more rows and/or one or more columns of k-space data, or an edge or interior region of the k-space image (containing pixels from multiple contiguous rows and columns, but not necessarily spanning the entirety of any particular row or column). The k-space data may be contiguous or separated into multiple spaced-apart (e.g., by one or more rows or columns) groups. It should be understood that image data can be acquired other than in discrete rows or columns, e.g., in tracks such as spirals or other patterns.

In step 230, partial image information corresponding to the portions of the image information identified in step 225 is newly acquired. Such partial image information typically corresponds to one or more partial k-space scans. For example, one or more MRI scans may be performed that collect image information corresponding to only a portion of the treatment area, e.g., the part(s) of the treatment area exhibiting the most rapid change. A partial k-space scan typically includes, consists essentially of, or consists of fewer rows and/or columns of k-space data than in a full k-space scan. In step 235, a new image is formed or acquired by replacing the portions identified in step 225 with the partial image information acquired in step 230; step 235 is therefore equivalent to updating the comparative image information (e.g., the most recent full k-space scan) with the newly acquired partial image data. In an optional step 240, the new image information is reconstructed to form an updated real-space image.

In some embodiments, the data from the previous image that is not updated is not re-used directly in the new image, but is instead adjusted on a pixel-by-pixel basis. For example, the intensities of pixels from the previous image that are not updated may be increased by a fraction of the mean intensity difference between pixels in the previous and new images that are updated. In another approach, the pixels that are not updated are treated as missing data and assigned values depending on a probability distribution in accordance, for example, with the EM algorithm.

In various embodiments, step 230 also includes newly acquiring one or more portions of the comparative image information not identified in step 225 as meeting a threshold of change, but acquiring such portions by sampling at a lower resolution. In this manner, the portions of the image information identified as not changing (at least, not undergoing change sufficiently rapid to warrant an updated scan) may be “spot-checked” to verify that they are still not changing or changing rapidly. In this manner, the benefits of a lower sampling rate may be obtained without re-analyzing full images, since in the “static” regions, a smaller number of pixels may be monitored over time and utilized for comparative purposes. In various embodiments, all or a portion of the lower-resolution sampled image information from the static regions may be incorporated into new images formed in step 235 and/or in images utilized for comparisons and identification of changing regions.

In various embodiments of the invention, steps 230 and 235 may be repeated one or more times during the course of the treatment (or a particular portion of a treatment sequence), thereby enabling high-speed imaging of the treatment target at a frequency comparable to, or even exceeding, the rate of one or more changes within the treatment area resulting from the treatment. As mentioned above, during a treatment sequence, one or more treatment parameters may be varied (i.e., step 210), and the method 200 may thus proceed from step 210 accordingly. In such cases, the most recently constructed image information (i.e., the new image information assembled in step 235) or the most recently acquired comparative full scan (i.e., in step 215) may be utilized as new baseline image information when the method is repeated, and new regions of change may be identified based thereon.

In various embodiments, the method 200 may proceed back to step 215, even without alteration of a treatment parameter, in order to acquire new comparative image information (e.g., a full k-space scan). In such embodiments, the new comparative image information may be compared (e.g., in step 225) to the original baseline image information acquired in step 205 and/or to prior comparative image information acquired in a previously completed step 215 and/or to new image information previously assembled in step 235.

In various embodiments of the invention, the treatment sequence may be altered based on the new image information developed in step 235 and/or on the real-space image reconstructed in step 240. For example, the ultrasound (or other therapeutic energy) beam may be steered during the treatment procedure to compensate for any target motion, or the beam intensity may be modulated in response to temperature changes in the target. Similarly, if changes in non-target organs or tissues are detected, such changes may be utilized to steer, shape, and/or modulate the beam so as to avoid or minimize their exposure to the therapeutic energy. Particularly, organs vulnerable to damage from the therapeutic beam are often of high interest, and any changes within or of such organs may be taken into account during beam-forming and/or steering such that the energy beam is shaped or modulated so as to treat the target while avoiding damaging temperature increases in sensitive adjacent organs.

As a conceptual example, consider a k-space image divided for update purposes into four regions A, B, C, D, and that it is desired to boost the overall imaging speed by a factor of two; accordingly, two rather than all four regions in each cycle. A representative workflow would begin with no prior information, and so sample:

t=0: A,B,C,D

where t identifies an imaging cycle.

At this stage the full k-space has been sampled, resulting in a baseline image. (Because this k-space image can be processed into the first real-space image, treatment may begin here.) Because there is no comparative information yet, two arbitrary regions are sampled at each cycle:

t=1: A,B

t=2: C,D

Now changes between pixels can be assessed. Assume, for example, that the pixels in region A are, on average, in changing significantly, B exhibits minor changes on average and regions C and D change negligibly. A representative sampling strategy is:

t=3: A,B

t=4: A,C

t=5: A,B

t=6: A,D

t=7: A,B

t=8: A,C

t=9: A,B

t=10: A,D

After each cycle the change in each region relative to the last time that region was sampled can be assessed. If, for example, region B is observed to change faster than region A, the sampling strategy can be switched dynamically to:

t=11: B,A

t=12: B,C

t=13: B,A

t=14: B,D

In this way, each region is updated at a frequency based on the observed need for updating, and there no need to do a full scan of all regions (e.g., in the middle of treatment) to adapt the sampling strategy.

At each cycle (t>1), the full k-space can be assembled from the recent measurements, e.g., after t=14 the full k-space is assembled from A13, B14, C12, D14 (where A13 refers to region A sampled at cycle 13). Alternatively, in some regions (e.g., region A), extrapolation or another estimation technique (e.g., probabilistic modeling using a Kalman filter) can be used to predict current pixel values at the region (rather than pixel) level, e.g., region A14 can be approximated by A13+(A13−A11)/2.

As described above, in some embodiments, imaging during a treatment procedure is used to quantitatively monitor changes of in vivo temperatures. This is particularly useful in MR-guided thermal therapy (e.g., MRgFUS treatment), where the temperature of a treatment area (e.g., a tumor to be destroyed by heat) should be continuously monitored in order to assess the progress of treatment and correct for local differences in heat conduction and energy absorption to avoid damage to tissues surrounding the treatment area. The monitoring (e.g., measurement and/or mapping) of temperature with MR imaging is generally referred to as MR thermometry or MR thermal imaging.

Among various methods available for MR thermometry, the proton resonance frequency (PRF) shift method is often the method of choice due to its excellent linearity with respect to temperature change, near-independence from tissue type, and temperature map acquisition with high spatial and temporal resolution. The PRF shift method is based on the phenomenon that the MR resonance frequency of protons in water molecules changes linearly with temperature (with a constant of proportionality that, advantageously, is relatively constant between tissue types). The frequency change with temperature is small, only −0.01 ppm/° C. for bulk water and approximately −0.0096 to −0.013 ppm/° C. in tissue, but the PRF shift is straightforwardly detected with a phase-sensitive imaging method in which image information acquired prior to a temperature change is compared to image information acquired after the temperature change, e.g., during or after treatment, thereby capturing a small phase change that is proportional to the change in temperature. A map of temperature changes may then be computed from the images by determining, on a pixel-by-pixel basis, phase differences between the baseline image and the treatment image, and converting the phase differences into temperature differences based on the PRF temperature dependence while taking into account imaging parameters such as the strength of the static magnetic field and echo time (TE) (e.g., of a gradient-recalled echo).

If the temperature distribution in the imaged area at the time of acquisition of the baseline image is known, the temperature-difference map may be added to that baseline temperature in order to obtain the absolute temperature distribution corresponding to the comparative image acquired during treatment. In some embodiments, the baseline temperature is simply uniform body temperature throughout the imaging region. More complicated baseline temperature distributions are, in some embodiments, determined prior to treatment by direct temperature-measurements in various locations in combination with interpolation and/or extrapolation based on a mathematical fit (e.g., a smooth, polynomial fit).

Thus, in various embodiments of the present invention, the identification of changing image portions performed in step 225 may include the processing of the acquired image information to form a temperature map corresponding to the treatment area. Portions of the treatment area experiencing changes in temperature exceeding a threshold may be identified and be the basis for, at least in part, the partial image information acquired in step 230.

Tracking and imaging methods in accordance herewith may be implemented using an (otherwise conventional) image-guided treatment system, such as the MRgFUS system 100 depicted in FIG. 1, in conjunction with a suitable image-processing and control facility (e.g., integrated with computation unit 112) in communication with the treatment apparatus (e.g., the beam former setting the phases and amplitudes of an ultrasound transducer array) and the imaging apparatus. In one embodiment, a tracking system 120 is implemented in the MRI apparatus 100 or is attached to the patient (as shown in FIG. 1) to provide information related to movement of the patient and/or absolute position within the patient. For example, a motion sensor (e.g., a respiration monitor belt) may be strapped around the patient's chest to provide information about motion of the patient; this information may then be used to initiate one or more steps of method 200. For example, if the movement of the patient exceeds a particular threshold, then step 215 of method 200 may be repeated more often to ensure that movement within the treatment area is properly accounted for in the partial image information acquired in step 230.

The image-processing and control facility of systems in accordance with embodiments of the invention may be implemented in any suitable combination of hardware, software, firmware, or hardwiring. FIG. 3 illustrates an exemplary embodiment where the facility is provided by a suitably programmed general-purpose computer 300. The computer includes a central processing unit (CPU) 302, system memory 304, and non-volatile mass storage devices 306 (such as, e.g., one or more hard disks and/or optical storage units). The computer 300 further includes a bidirectional system bus 308 over which the CPU 302, memory 304, and storage devices 306 communicate with each other and with internal or external input/output devices, such as traditional user interface components 310 (including, e.g., a screen, a keyboard, and a mouse) as well as the treatment apparatus 312, the imaging apparatus 314, and (optionally) any temperature sensors 316 facilitating absolute-temperature measurements.

The system memory 304 contains instructions, conceptually illustrated as a group of modules, that control the operation of CPU 302 and its interaction with the other hardware components. An operating system 318 directs the execution of low-level, basic system functions such as memory allocation, file management and operation of mass storage devices 306. At a higher level, one or more service applications provide the computational functionality required for image-processing, tracking, and (optionally) thermometry. For example, as illustrated, the system may include an image-reconstruction module 320 for reconstructing real-space images from raw image data received from the imaging apparatus 314 and for constructing full images (e.g., in k-space) from portions of baseline and/or comparative image information in combination with partial image information related to changing portions of the treatment area. The system may also include an image comparison module 322 for measuring similarity and/or dissimilarity between baseline and comparative images (whether raw data such as k-space data or reconstructed images). The image analysis module 324 extracts information (e.g., locational and/or temperature information of the target and/or other obj ect(s) of interest) from the images acquired or reconstructed as described above. In addition, the system may include a beam-adjustment module 326 for computing phase shifts or other parameters of the treatment apparatus to compensate for any detected changes in the treatment area, and a thermal-map module 328 that subtracts baseline from comparative treatment images to obtain a temperature difference map and, if the absolute temperature corresponding to the selected baseline image is known, an absolute-temperature map for the comparative treatment image and/or images constructed using partial images. The various modules may be programmed in any suitable programming language, including, without limitation, high-level languages such as C, C++, C#, Ada, Basic, Cobra, Fortran, Java, Lisp, Perl, Python, Ruby, or Object Pascal, or low-level assembly languages; in some embodiments, different modules are programmed in different languages.

### EXAMPLE

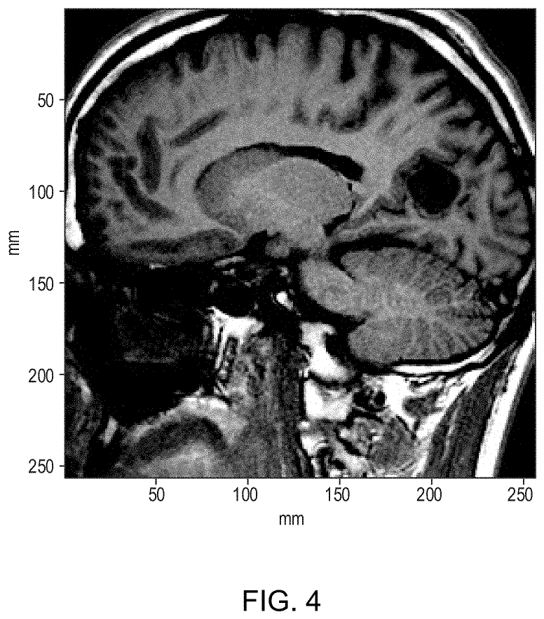

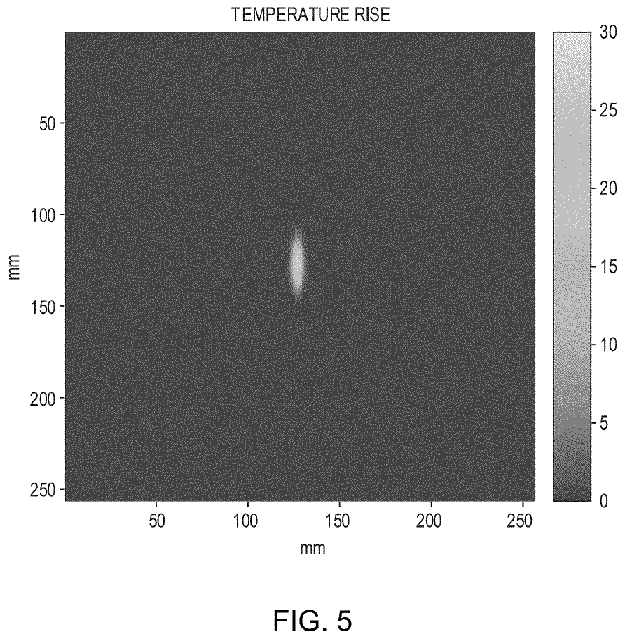

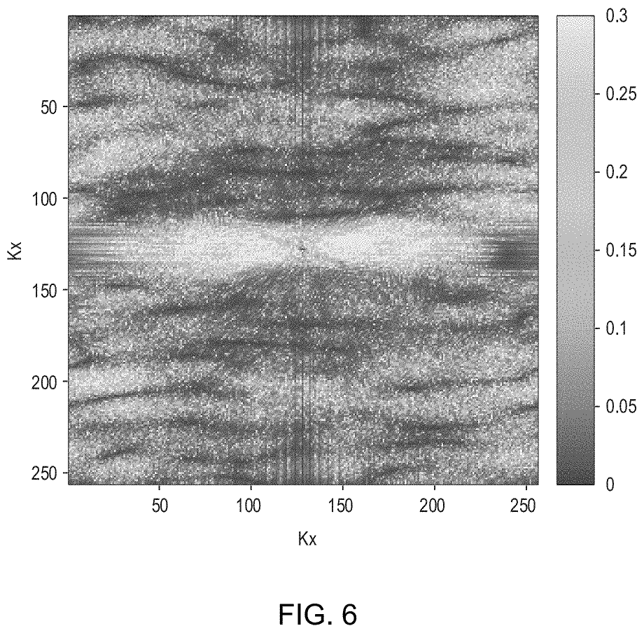

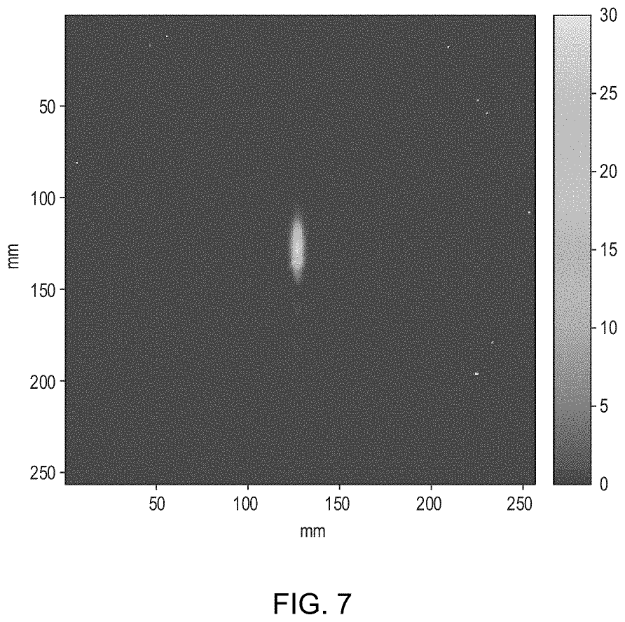

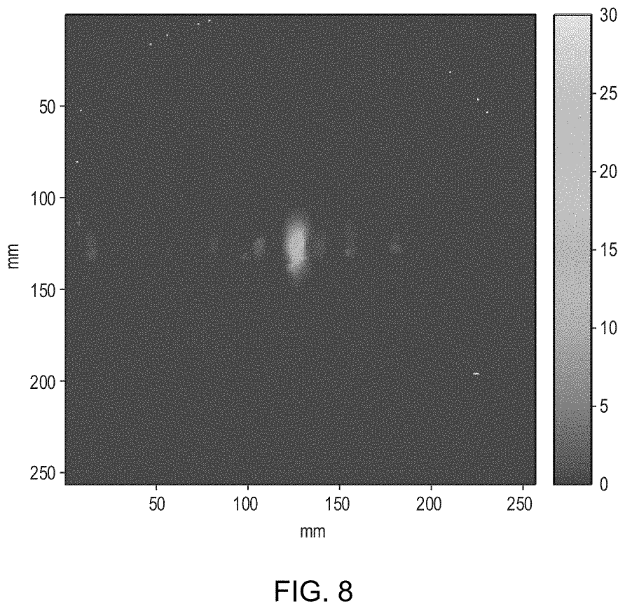

FIG. 4 is an Mill real-space image of a human patient prior to undergoing treatment via application of focused ultrasound energy. The image in FIG. 4 depicts a cross-sectional slice of the brain of the patient. FIG. 5 is a temperature map constructed from an MRI scan acquired after the heating of a target within the patient via application of focused ultrasound energy. As shown, the temperature rise is limited to a small portion of the treatment area and has an approximately elliptical shape. The temperature rise at the center of the target is approximately 30° C., and the temperature rise diminishes approximately radially from the center of the target. FIG. 6 is a graph in k-space corresponding to the temperature change presented in the temperature map of FIG. 5. FIG. 6 graphically presents the normalized value of the relative change between the k-space representation of FIG. 4 after treatment (i.e., the heat pattern of FIG. 5 applied to the image in FIG. 4) and the k-space representation of the baseline acquired from FIG. 4 prior to treatment; the relative change represented in FIG. 6 is capped at 30% change in complex k-space. As shown, the region of largest change revealed in FIG. 6 falls approximately within rows 110-140 of the image. Thus, only partial images of that portion of the treatment area were acquired in order to increase throughput of the imaging and temperature detection. FIG. 7 depicts a newly calculated temperature map obtained from a constructed image based on the k-space representation of FIG. 4 along with newly sampled data falling only within rows 110-140 of the k space. As shown, the calculated temperature map in FIG. 7 compares quite favorably to the image of FIG. 5 and was acquired and constructed in a fraction of the time required to construct the image of FIG. 4. FIG. 8 depicts a calculated temperature map which was calculated from a reconstructed image based on the k-space representation of FIG. 4 in combination with newly sampled data falling only within columns 110-140 of the k-space image rather than rows 110-140. As shown, this reconstruction does not adequately capture the change, and the image does not compare favorably to FIG. 5. Thus, in various embodiments of the present invention, selection of full rows of k-space for acquisition of partial image information may yield superior results.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A system for imaging a target region comprising a feature therewithin, the system comprising:
an imaging apparatus, operable in conjunction with a treatment apparatus, for acquiring images, the imaging apparatus being configured to acquire and computationally store (i) a baseline k-space image of the target region, (ii) a comparative k-space image of the target area during an operational sequence, and (iii) one or more new k-space images each encompassing only a portion of the target region during the operational sequence; and
a computation unit configured to (i) computationally compare the comparative k-space image with the baseline k-space image to identify one or more first image regions of the k space associated with a changed characteristic within the target region, the comparative k-space image comprising (a) the one or more first image regions and (b) a remaining image region, and (ii) cause the imaging apparatus to acquire a new k-space image by sampling only in the one or more first image regions.

2. The system of claim 1, wherein the new k-space image comprises pixels corresponding to the newly sampled one or more image regions and information based at least in part on previously sampled pixels corresponding to the remaining image region.

3. The system of claim 1, wherein the computation unit is configured to computationally reconstruct a real-space image from the comparative k-space image.

4. The system of claim 1, wherein the computation unit is configured to computationally reconstruct a real-space image from the new k-space image.

5. The system of claim 1, wherein the imaging apparatus is an MRI apparatus.

6. The system of claim 1, wherein the treatment apparatus comprises one or more ultrasound transducers.

7. The system of claim 1, the changed characteristic within the target region comprises a pixel value.

8. The system of claim 1, wherein the computational unit is configured to steer and/or modulate an energy beam based on the new k-space image and/or a real-space image computationally reconstructed therefrom.

9. The system of claim 8, wherein the energy beam is a focused ultrasound beam.

10. The system of claim 1, wherein the operational sequence comprises exposure of a target other than the feature, the computational unit being configured to shape and/or steer an energy beam onto the target so as to avoid the feature based on the new k-space image and/or a real-space image computationally reconstructed therefrom.

11. The system of claim 10, wherein the energy beam is a focused ultrasound beam.

12. The system of claim 1, wherein the computation unit is configured to (i) identify a plurality of first image regions of the k space associated with the changed characteristic within the target area, and (ii) sample in the first image regions at a frequency, for each first image region, based at least in part on a magnitude of the change in the characteristic therein.

13. The system of claim 1, wherein the one or more first image regions are identified at least in part by estimation based on at least one previous k-space image.

14. The system of claim 1, wherein the computation unit is configured to iteratively repeat the acquisition step by sampling in the one or more first image regions at a first frequency and in the remaining region at a second frequency lower than the first frequency.

15. A method for imaging, during an operational sequence, a target region comprising a feature therewithin, the method comprising:
acquiring a baseline k-space image of the target region; and
thereafter, during the operational sequence:
(a) acquiring a comparative k-space image of the target region;
(b) computationally comparing the comparative k-space image with the baseline k-space image to identify one or more first image regions of the comparative k-space image having a changed characteristic, the comparative k-space image comprising (i) the one or more first image regions and (ii) a remaining image region; and
(c) subsequently acquiring a new k-space image by sampling only the one or more first image regions, the new k-space image comprising pixels corresponding to the newly sampled one or more first image regions and additional pixel values based at least in part on previously sampled pixels corresponding to the remaining image region.

16. The method of claim 15, further comprising displaying a real-space image computationally reconstructed from the new k-space image.

17. The method of claim 15, wherein step (c) is repeated one or more times.

18. The method of claim 17, further comprising repeating steps (a) and (b) after repeating step (c) one or more times.

19. The method of claim 15, wherein the changed characteristic within the target region is a pixel value.

20. The method of claim 15, wherein the baseline k-space image and the comparative k-space image are full-scan MRI images.

21. The method of claim 15, wherein the new k-space image is a partial-scan MRI image.

22. The method of claim 15, wherein the operational sequence comprises exposure of the feature to an energy beam.

23. The method of claim 15, wherein the operational sequence comprises steering and/or modulating an energy beam based on the new k-space image and/or a real-space image computationally reconstructed therefrom.

24. The method of claim 23, wherein the energy beam is a focused ultrasound beam.

25. The method of claim 15, wherein the operational sequence comprises exposure of a target other than the feature.

26. The method of claim 25, further comprising shaping and/or steering an energy beam onto the target region so as to avoid the feature based on the new k-space image and/or a real-space image computationally reconstructed therefrom.

27. The method of claim 26, wherein energy beam is a focused ultrasound beam.

28. The method of claim 15, wherein a plurality of first image regions of the k space associated with the changed characteristic within the target area is identified, and further comprising the step of sampling in the first image regions at a frequency, for each first image region, based at least in part on a magnitude of the change in the characteristic therein.

29. The method of claim 15, wherein the one or more first image regions are identified at least in part by estimation based on at least one previous k-space image.

30. The method of claim 15, wherein step (c) is iteratively repeated by sampling in the one or more first image regions at a first frequency and in the remaining region at a second frequency lower than the first frequency.
